(19) United States
(12) Patent Application Publication
(10) Pub. No.: US 2006/0263315 A1
(43) Pub. Date: Nov. 23, 2006

(54) Composition comprising an electrophilic monomer and an amino acid other than cysteine, and cosmetic treatment processes

(76) Inventors: Gregory Plos, Tokyo (JP); Gaelle Brun, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/393,738

(22) Filed: Mar. 31, 2006

Related U.S. Application Data
(60) Provisional application No. 60/669,891, filed on Apr. 11, 2005.

(30) Foreign Application Priority Data
Mar. 31, 2005 (FR) 05 03152

(51) Int. Cl. A61K 8/81

(52) U.S. Cl. 424/70.7

(57) ABSTRACT

The present invention relates to a fluid composition comprising, in a cosmetically acceptable medium, at least one electrophilic monomer and at least one amino acid other than cysteine. The invention also relates to a treatment process using this composition and to its use for giving keratin fibres a conditioning effect and/or a styling effect.

This application claims benefit of U.S. Provisional Application No. 60/669,891, filed Apr. 11, 2005, the contents of which are incorporated herein by reference. This application also claims benefit of priority under 35 U.S.C. §119 to French Patent Application No. FR 05 03152, filed Mar. 31, 2005, the contents of which are also incorporated herein by reference.

The present invention relates to a composition comprising, in a cosmetically acceptable medium, at least one electrophilic monomer and at least one amino acid other than cysteine, to its use for the cosmetic treatment of keratin fibres and to a cosmetic treatment process using such a composition.

In the field of cosmetics, it is sought to modify the surface properties of keratin fibres such as the hair, for example to give the hair a conditioning effect such as softness or sheen. To do this, cosmetic compositions based on conditioning agents such as silicones or polymers that have high affinity for keratin fibres, in particular for the hair, are generally used.

However, these conditioning agents have a tendency to be removed in the course of washing with shampoo, making it necessary to renew applications of the compositions on the hair.

To increase the remanence of a polymer deposit, it may be envisaged to perform a radical polymerization of certain monomers directly on the hair. However, substantial degradation of the hair fibres is observed, probably associated with the polymerization initiators, and the hair thus treated is difficult to disentangle.

The Applicant has found, surprisingly, that by combining at least one amino acid other than cysteine with at least one electrophilic monomer as described below, in a cosmetically acceptable medium, it is possible to obtain improved, long-lasting conditioning and sheen of the hair.

Specifically, the application of a composition comprising such a combination leads to the formation of a shiny coating or covering that is remanent, especially with respect to shampooing, even after washing the head of hair several times. The covering obtained can give, in addition to the sheen and conditioning properties, pronounced remanent styling properties.

In addition, surprisingly, the hair remains perfectly individualized and can be styled without problem.

One subject of the invention is thus a fluid composition comprising, in a cosmetically acceptable medium, at least one electrophilic monomer as described below and at least one amino acid other than cysteine.

Another subject of the present invention consists of the use of the said composition for the cosmetic treatment of keratin fibres, and more particularly the hair.

A subject of the invention is also a cosmetic process for treating keratin fibres, and more particularly the hair, using the said composition.

Other subjects, characteristics, aspects and advantages of the invention will emerge even more clearly on reading the description and the example that follows.

According to the invention, the fluid composition comprises, in a cosmetically acceptable medium, at least one electrophilic monomer and at least one amino acid other than cysteine, the said electrophilic monomer corresponding to the general formula (A): 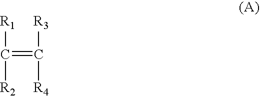
in which:

R₁ and R₂ denote, independently of each other, a sparingly- or non-electro-attractive group (sparingly or non-inductive-attractive) such as:
a hydrogen atom,
a saturated or unsaturated, linear, branched or cyclic hydrocarbon-based group preferably containing from 1 to 20 and better still from 1 to 10 carbon atoms, and optionally containing one or more nitrogen, oxygen or sulfur atoms, and optionally substituted with one or more groups chosen from —OR, —COOR, —COR, —SH, —SR and —OH, and halogen atoms,
a modified or unmodified polyorganosiloxane residue,
a polyoxyalkylene group,

R₃ and R₄ denote, independently of each other, an electro-attractive (or inductive-attractive) group preferably chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups such as phenyl, and aryloxy groups such as phenoxyloxy,

R denotes a saturated or unsaturated, linear, branched or cyclic hydrocarbon-based group preferably containing from 1 to 20 and better still from 1 to 10 carbon atoms, and optionally containing one or more nitrogen, oxygen or sulfur atoms, and optionally substituted with one or more groups chosen from —OR′, —COOR′, —COR′, —SH, —SR′ and —OH, halogen atoms, and a polymer residue that may be obtained by radical polymerization, by polycondensation or by ring opening, R′ denoting a C₁-C₁₀ alkyl group.

For the purposes of the present invention, the term “fluid composition” means a composition having a viscosity of less than 5 Pa·s (50 poises), measured at 25° C. and at a shear rate of 1 s⁻¹, with a Rheomat 180 viscometer from the company Mettler or with an RS600 viscometer from the company Thermo.

The viscosity is preferably in the range from 0.01 to 5 Pa·s (10 centipoises to 50 poises), better still from 0.1 to 4 Pa·s (100 centipoises to 40 poises) and even more preferentially from 0.2 to 2 Pa·s (2 to 20 poises).

The term “cosmetically acceptable medium” means a medium that is compatible with keratin fibres such as the hair.

The cosmetically acceptable medium is preferably anhydrous.

The term “anhydrous medium” means a medium containing less than 1% by weight of water relative to the total weight of the composition.

The cosmetically acceptable medium is preferably chosen from organic oils; silicones such as volatile silicones, amino or non-amino silicone gums or oils and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil and lemon oil; waxes; or alternatively organic compounds such as C₅-C₁₀ alkanes, acetone, methyl ethyl ketone, esters of C₁-C₂₀ acids and of C₁-C₈ alcohols such as methyl acetate, butyl acetate, ethyl acetate and isopropyl myristate, dimethoxyethane, diethoxyethane, C₁₀-C₃₀ fatty alcohols such as lauryl alcohol, cetyl alcohol, stearyl alcohol and behenyl alcohol; C₁₀-C₃₀ fatty acids such as lauric acid and stearic acid; C₁₀-C₃₀ fatty amides such as lauric diethanolamide, and C₁₀-C₃₀ fatty alkyl esters such as C₁₀-C₃₀ fatty alkyl benzoates, and mixtures thereof.

Preferably, the organic compounds constituting the medium are chosen from compounds that are liquid at a temperature of 25° C. and at 10⁵ Pa (760 mmHg).

The cosmetically acceptable anhydrous medium preferably used is chosen from olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil; polybutene oil, isononyl isononanoate, isostearyl malate, pentaerythrityl tetraisostearate, tridecyl trimellitate, and a mixture of cyclopentasiloxane (14.7% by weight)/polydimethylsiloxane dihydroxylated in the α and ω positions (85.3% by weight), or mixtures thereof.

The term “amino acids” means a compound which is not obtained by polycondensation of identical or different amino acids.

The amino acids that may be used according to the invention comprise at least one amine function and at least one acid function. The acid function(s) may be carboxylic, sulfonic, phosphonic or phosphoric, and are preferably carboxylic. 27

Preferably, the amino acids used in the present invention are α-amino acids, i.e. they comprise an amine function and a group R in the a position relative to the acid function. They may be represented by the following formula: 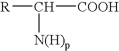
in which:

when p=2, R represents a hydrogen atom, an aliphatic group optionally containing a heterocyclic portion, or an aromatic group,

or

when p=1, R can form a heterocycle with the nitrogen atom of —N(H)ₚ. This heterocycle is preferably a saturated 5-membered ring, optionally substituted with one or more C₁₋₄ alkyl or hydroxyl groups.

Preferably, the aliphatic group is a linear or branched C₁₋₄ alkyl group; a linear or branched C₁₋₄ hydroxyalkyl group; a linear or branched C₁₋₄ aminoalkyl group; a linear or branched (C₁₋₄ alkyl)thio(C₁₋₄)alkyl group; a linear or branched C₂₋₄ carboxyalkyl group; a linear or branched ureidoalkyl group, a linear or branched guanidinoalkyl group, a linear or branched imidazoloalkyl group or a linear or branched indolylalkyl group, the alkyl portions of these last four groups comprising from one to four carbon atoms.

Preferably, the aromatic group is a C₆ aryl or C₇₋₁₀ aralkyl group, the aromatic nucleus optionally being substituted with one or more C₁₋₄ alkyl or hydroxyl groups.

As amino acids that may be used in the present invention, mention may be made especially of aspartic acid, glutamic acid, alanine, arginine, asparagine, glutamine, glycine, histidine, isoleucine, leucine, lysine, methionine, N-phenylalanine, proline, serine, threonine, tryptophan, tyrosine, valine and hydroxyproline.

Preferentially, the amino acids comprise only one amine function, which may or may not be engaged in a ring, and only one acid function. The amino acids that are particularly preferred in the present invention are alanine, asparagine, glutamine, glycine, isoleucine, leucine, methionine, N-phenylalanine, proline, serine, threonine, tyrosine and valine.

The compositions used in accordance with the invention generally have an amino acid concentration of between 0.1% and 20% by weight, preferably between 0.5% at 15% by weight and more particularly between 1% and 10% by weight relative to the total weight of the composition.

The term “electrophilic monomer” means a monomer capable of polymerizing via anionic polymerization in the presence of a nucleophilic agent, for instance the hydroxide ions (OH⁻) contained in water at neutral pH.

The term “anionic polymerization” means the mechanism defined in the book “Advanced Organic Chemistry”, Third Edition, by Jerry March, pages 151 to 161.

The electrophilic monomers used in accordance with the invention are the monomers of structure: 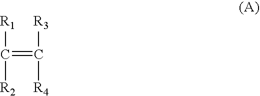
in which:

R₁ and R₂ denote, independently of each other, a sparingly- or non-electro-attractive group (sparingly or non-inductive-attractive) such as:
a hydrogen atom,
a saturated or unsaturated, linear, branched or cyclic hydrocarbon-based group preferably containing from 1 to 20 and better still from 1 to 10 carbon atoms, and optionally containing one or more nitrogen, oxygen or sulfur atoms, and optionally substituted with one or more groups chosen from —OR, —COOR, —COR, —SH, —SR and —OH, and halogen atoms,
a modified or unmodified polyorganosiloxane residue,
a polyoxyalkylene group,

R₃ and R₄ denote, independently of each other, an electro-attractive (or inductive-attractive) group preferably chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups such as phenyl, and aryloxy groups such as phenoxyloxy,

R denotes a saturated or unsaturated, linear, branched or cyclic hydrocarbon-based group preferably containing from 1 to 20 and better still from 1 to 10 carbon atoms, and optionally containing one or more nitrogen, oxygen or sulfur atoms, and optionally substituted with one or more groups chosen from —OR′, —COOR′, —COR′, —SH, —SR′ and —OH, halogen atoms, and a polymer residue that may be obtained by radical polymerization, by polycondensation or by ring opening, R′ denoting a C₁-C₁₀ alkyl group.

The term “electro-attractive or inductive-attractive group (—I)” means any group that is more electronegative than carbon. Reference may be made to the publication P. R. Wells, Prog. Phys. Org. Chem., Vol 6, 111 (1968).

The term “sparingly or non-electro-attractive group” means any group whose electronegativity is less than or equal to that of carbon.

The alkenyl or alkynyl groups preferably contain from 2 to 20 carbon atoms and better still from 2 to 10 carbon atoms.

As saturated or unsaturated, linear, branched or cyclic hydrocarbon-based groups preferably containing from 1 to 20 carbon atoms and better still from 1 to 10 carbon atoms, mention may be made especially of linear or branched alkyl, alkenyl or alkynyl groups, such as methyl, ethyl, n-butyl, tert-butyl, isobutyl, pentyl, hexyl, octyl, butenyl or butynyl; cycloalkyl or aromatic groups.

Examples of substituted hydrocarbon-based groups that may be mentioned include hydroxyalkyl and polyhaloalkyl groups.

Examples of unmodified polyorganosiloxanes that may especially be mentioned include polyalkylsiloxanes such as polydimethylsiloxanes, polyarylsiloxanes such as polyphenylsiloxanes, and polyarylalkylsiloxanes such as polymethylphenylsiloxanes.

Among the modified polyorganosiloxanes that may especially be mentioned are polydimethylsiloxanes containing polyoxyalkylene and/or siloxy and/or silanol and/or amine and/or imine and/or fluoroalkyl groups.

Among the polyoxyalkylene groups that may especially be mentioned are polyoxyethylene groups and polyoxypropylene groups preferably containing 1 to 200 oxyalkylene units.

Among the mono- or polyfluoroalkyl groups that may especially be mentioned are groups such as —(CH₂)ₙ—(CF₂)ₘ—CF₃ or —(CH₂)ₙ—(CF₂)ₘ—CHF₂ with n=1 to 20 and m=1 to 20.

The substituents R₁ to R₄ may optionally be substituted with a group having cosmetic activity. The cosmetic activities that are particularly used are obtained from groups having colouring, antioxidant, UV-screening and conditioning functions.

As examples of groups having a colouring function, mention may be made especially of azo, quinone, methine, cyanomethine and triarylmethane groups.

As examples of groups having an antioxidant function, mention may be made especially of groups of butylhydroxyanisole (BHA), butylhydroxytoluene (BHT) or vitamin E type.

As examples of groups having a UV-screening function, mention may be made especially of groups of the benzophenone, cinnamate, benzoate, benzylidenecamphor and dibenzoylmethane type.

As examples of groups having a conditioning function, mention may be made especially of cationic groups and groups of fatty ester type.

Among the monomers mentioned above, the ones that are preferred are the monomers of the cyanoacrylate family and the derivatives thereof of formula (B):

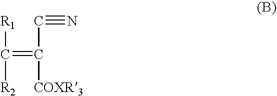

X denoting NH, S or O,

R₁ and R₂ having the same meanings as above,

R′₃ possibly denoting a hydrogen atom or a group R as defined for formula (A).

Preferably, X denotes O.

Compounds of formula (B) that may be mentioned include the monomers:
a) belonging to the family of polyfluoro(C₁₋₂₀)alkyl 2-cyanoacrylates such as: the ester 2,2,3,3-tetrafluoropropyl 2-cyano-2-propenoate of formula:

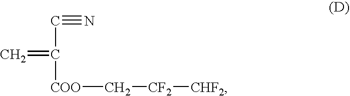

or the ester 2,2,2-trifluoroethyl 2-cyano-2-propenoate of formula:

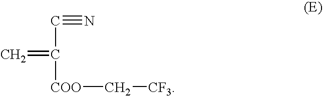

b) the C₁-C₁₀ alkyl or (C₁-C₄ alkoxy)(C₁-C₁₀ alkyl) cyanoacrylates.

Mention may be made more particularly of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

In the context of the invention, it is preferred to use the monomers b).

The monomers that are the most particularly preferred are those of formula F and mixtures thereof: 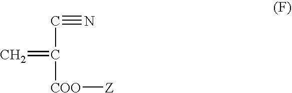
in which: Z=-(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃,
—(CH₂)₄—CH(C₂H₅)—CH₃.

The monomers used in accordance with the invention may be covalently bonded to supports such as polymers, oligomers or dendrimers. The polymer or the oligomer may be linear, branched, in comb form or in block form. The distribution of the monomers of the invention over the polymeric, oligomeric or dendritic structure may be random, in an end position or in the form of blocks.

The compositions used in accordance with the invention generally have a concentration of electrophilic monomer according to the invention of between 0.001% and 80% by weight, preferably between 0.002% and 75% by weight, more particularly between 0.1% and 40% by weight and even more preferentially between 1% and 20% by weight relative to the total weight of the composition.

Polymerization inhibitors and more particularly anionic and/or radical polymerization inhibitors may also be introduced into the compositions, in order to enhance the stability of the composition over time. In a non-limiting manner, the following polymerization inhibitors may be mentioned: sulfur dioxide, nitric oxide, lactone, boron trifluoride, hydroquinone and derivatives thereof such as hydroquinone monoethyl ether, tert-butylhydroquinone (TBHQ), benzoquinone and derivatives thereof such as duroquinone, catechol and derivatives thereof such as t-butylcatechol and methoxycatechol, anisole and derivatives thereof such as methoxyanisole, hydroxyanisole or butylhydroxyanisole, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans and 3-sulfonene, and mixtures thereof. The alkyl groups preferably denote groups containing 1 to 6 carbon atoms.

It is also possible to use mineral or organic acids, the latter containing one or more carboxylic or sulfonic groups, with a pKa of between 0 and 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di- or trichloroacetic acid, salicylic acid and trifluoroacetic acid.

The amount of inhibitor may range from 10 ppm to 20%, more preferentially from 10 ppm to 5% and even more preferentially from 10 ppm to 1% by weight relative to the total weight of the composition.

The compositions in accordance with the invention may also contain at least one agent usually used in cosmetics, chosen, for example, from reducing agents, fatty substances, plasticizers, softeners, antifoams, moisturizers, pigments, clays, mineral fillers, UV-screening agents, mineral colloids, peptizers, solubilizers, fragrances, preserving agents, anionic, cationic, nonionic or amphoteric surfactants, fixing or non-fixing polymers, polyols, proteins, vitamins, direct or oxidation dyes, nacreous agents, propellants, and mineral or organic thickeners such as benzylidenesorbitol and N-acylamino acids.

These agents or the amino acid(s) may optionally be encapsulated. The capsule may be of polycyanoacrylate type.

The composition is preferably used on the keratin fibres, such as the hair, preferably in the presence of a nucleophilic agent, for cosmetic treatment thereof.

It is especially used to give a conditioning effect such as softness; disentangling; sheen; volume, and/or a styling effect such as hair hold.

A cosmetic treatment process according to the invention comprises the application of a composition as defined above to keratin fibres such as the hair, preferably in the presence of a nucleophilic agent as defined below.

The nucleophilic agents capable of initiating anionic polymerization are systems that are known per se, which are capable of generating a carbanion on contact with a nucleophilic agent, such as the hydroxide ions contained in water at neutral pH. The term “carbanion” means the chemical species defined in “Advanced Organic Chemistry”, third edition, by Jerry March, page 141.

The nucleophilic agents may consist of a molecular compound, an oligomer, a dendrimer or a polymer containing nucleophilic functions. In a non-limiting manner, nucleophilic functions that may be mentioned include the following functions: R₂N⁻, NH₂⁻, Ph₃C⁻, R₃C⁻, PhNH⁻, pyridine, ArS⁻, R—C≡C⁻, RS⁻, SH, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, Ph representing a phenyl group; Ar representing an aryl group and R representing a C₁-C₁₀ alkyl group.

Preferably, the nucleophilic agent is water. This water may be provided by wetting beforehand.

It is also possible, in order to modify the reaction kinetics, to moisten the keratin fibres such as the hair beforehand using an aqueous solution whose pH has been adjusted using a base, an acid or an acid/base mixture. The acid and/or the base may be mineral or organic.

It is also possible to modify the anionic polymerization kinetics by preimpregnating the keratin fibres such as the hair with a nucleophilic agent other than water. The nucleophilic agent may be used pure, as a solution or in the form of an emulsion, or may be encapsulated.

To modify the anionic polymerization kinetics, it is also possible to increase the nucelophilicity of the keratin fibres such as the hair via chemical conversion of the keratin material.

Examples of chemical conversions that may be mentioned include the reduction of the disulfide bridges of which keratin is partly composed, into thiols, before applying the composition of the invention. In a non-exhaustive manner, as reducing agents for the disulfide bridges of which keratin is partly composed, mention may be made of the following compounds:

anhydrous sodium thiosulfate,

powdered sodium metabisulfite,

thiourea,

ammonium sulfite,

thioglycolic acid,

thiolactic acid,

ammonium thiolactate,

glyceryl monothioglycolate,

ammonium thioglycolate,

thioglycerol,

2,5-dihydroxybenzoic acid,

diammonium dithioglycolate,

strontium thioglycolate,

calcium thioglycolate,

zinc formosulfoxylate,

isooctyl thioglycolate,

di-cysteine,

monoethanolamine thioglycolate.

To modify the anionic polymerization kinetics, and more specifically to reduce the rate of polymerization of the monomers of the invention, it is possible to increase the viscosity of the composition. To do this, one or more polymers that have no reactivity towards the monomers in accordance with the invention may be added to the composition of the invention. In this context, mention may be made, in a non-exhaustive manner, of poly(methyl methacrylate) (PMMA) or alternatively cyanoacrylate-based copolymers as described in U.S. Pat. No. 6,224,622.

In order to improve, inter alia, the adhesion of the poly(cyanoacrylate) formed in situ, the fibre may be pretreated with polymers of any type, or a haircare treatment may be performed before applying the composition of the invention, for instance a direct dyeing or oxidation dyeing, permanent-waving or hair relaxing operation.

The application of the compositions as described above may or may not be followed by rinsing.

The compositions may be in the form of a lotion, a spray or a mousse, and may be applied as a shampoo or a hair conditioner.

Another subject of the invention consists of a cosmetic process for treating keratin fibres, comprising at least two steps, one step comprising the application of at least one amino acid other than cysteine as defined above, and, with or without intermediate rinsing, another step comprising the application of at least one electrophilic monomer as defined above, the order of the steps being unimportant.

One particular embodiment of the invention consists of the application of at least one amino acid other than cysteine before the application of at least one electrophilic monomer.

Another particular embodiment of the invention consists of the application of at least one electrophilic monomer as defined above before the application of at least one amino acid other than cysteine.

One preferred embodiment of the invention consists of a process comprising the steps consisting in:
(1) applying to the hair an aqueous solution containing 0.05-40% by weight, preferably 0.1-35% by weight and better still 0.25-25% by weight of amino acid(s), the percentages being expressed relative to the total weight of the solution,
(2) applying to the hair, after optional intermediate rinsing, a composition comprising 0.001-80% by weight, preferably 0.1% to 40% by weight and better still 1% to 20% by weight of electrophilic monomer(s) as defined above, the percentages being expressed relative to the total weight of the composition.

The order of the two steps (1) and (2) may be inverted.

The application of a cosmetic product may also precede the first step or follow the second step. Each step may also be interrupted by rinsing and optionally by drying, the drying possibly being performed using a hood, a hairdryer and/or a smoothing iron.

The steps of the processes described above may be repeated so as to obtain several layers depending on the type of deposit desired in terms of chemical nature, mechanical strength, thickness, appearance and/or feel.

A subject of the invention is also a kit comprising a first composition containing at least one electrophilic monomer as defined above and optionally at least one anionic and/or radical polymerization inhibitor as defined above, and also a second composition comprising, in a cosmetically acceptable medium, at least one amino acid other than cysteine, as defined above.

The example that follows is given as an illustration of the present invention.

In the example that follows, all the amounts are indicated as weight percentages of active material relative to the total weight of the composition, unless otherwise indicated.

### EXAMPLE

A composition A comprising the following ingredients was prepared, the proportions being indicated as weight percentages.

| | |
|---|---|
| 2-Octyl 2-cyanoacrylate | 10% |
| 50/50 by weight mixture of DC 1501 Fluid (Dow Corning) | 90% |
| (poly(α,ω- | |
| dihydroxyldimethylsiloxane/cyclopentadimethylsiloxane) | |
| (14.7/85.3)) and DC 245 Fluid (Dow Corning) | |
| (cyclopentadimethylsiloxane) | |

A 1 -g lock of natural chestnut-brown hair was moistened with 0.5 g of an aqueous solution containing 0.2 mol/l of glycine.

0.16 g of composition A was then applied to the lock.

After a leave-on time of 15 minutes, the lock was dried for two minutes with a hairdryer. The lock obtained is shinier than a lock that has been moistened with 0.5 g of water instead of the glycine solution.

What is claimed is:

1. Fluid composition comprising, in a cosmetically acceptable medium, at least one electrophilic monomer and at least one amino acid other than cysteine, the said electrophilic monomer corresponds to the formula: 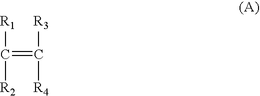
in which:
R₁ and R₂ denote, independently of each other, a sparingly- or non-electron-attractive group chosen from:
a hydrogen atom,
a saturated or unsaturated, linear, branched or cyclic hydrocarbon-based group containing from 1 to 20 carbon atoms, and optionally containing one or more nitrogen, oxygen or sulfur atoms, and optionally substituted with one or more groups chosen from —OR, —COOR, —COR, —SH, —SR and —OH, and halogen atoms,
a modified or unmodified polyorganosiloxane residue,
a polyoxyalkylene group,
R₃ and R₄ denote, independently of each other, an electro-attractive group chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups and aryloxy groups,
R denotes a saturated or unsaturated, linear, branched or cyclic hydrocarbon-based group containing from 1 to 20 carbon atoms, and optionally containing one or more nitrogen, oxygen or sulfur atoms, and optionally substituted with one or more groups chosen from —OR′, —COOR′, —COR′, —SH, —SR′ and —OH, halogen atoms, and a polymer residue that may be obtained by radical polymerization, by polycondensation or by ring opening, R′ denoting a C₁-C₁₀ alkyl group.

2. Composition according to claim 1, characterized in that the amino acid comprises a carboxylic acid function.

3. Composition according to claim 1 or 2, characterized in that the amino acid is an α-amino acid.

4. Composition according to any one of the preceding claims, characterized in that the amino acid corresponds to the formula: 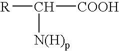
in which:
when p=2, R represents a hydrogen atom, an aliphatic group optionally containing a heterocyclic portion, or an aromatic group,
or
when p=1, R can form a heterocycle with the nitrogen atom of —N(H)ₚ.

5. Composition according to claim 4, characterized in that the aliphatic group is a linear or branched C₁₋₄ alkyl group; a linear or branched C₁₋₄ hydroxyalkyl group; a linear or branched C₁₋₄ aminoalkyl group; a linear or branched (C₁₋₄ alkyl)thio(C₁₋₄)alkyl group; a linear or branched C₂₋₄ carboxyalkyl group; a linear or branched ureidoalkyl group, a linear or branched guanidinoalkyl group, a linear or branched imidazoloalkyl group or a linear or branched indolylalkyl group, the alkyl portions of these last four groups comprising from one to four carbon atoms.

6. Composition according to claim 4, characterized in that the aromatic group is a C₆ aryl or C₇₋₁₀ aralkyl group, the aromatic nucleus optionally being substituted with one or more C₁₋₄ alkyl or hydroxyl groups.

7. Composition according to claim 4, characterized in that the heterocycle, when p=1, is a saturated 5-membered ring, optionally substituted with one or more C₁₋₄ alkyl or hydroxyl groups.

8. Composition according to any one of the preceding claims, characterized in that the said amino acid is chosen from aspartic acid, glutamic acid, alanine, arginine, asparagine, glutamine, glycine, histidine, isoleucine, leucine, lysine, methionine, N-phenylalanine, proline, serine, threonine, tryptophan, tyrosine, valine and hydroxyproline.

9. Composition according to claim 8, characterized in that the said amino acid is chosen from alanine, asparagine, glutamine, glycine, isoleucine, leucine, methionine, N-phenylalanine, proline, serine, threonine, tyrosine and valine.

10. Composition according to any one of the preceding claims, characterized in that the said amino acid(s) is (are) present in a concentration of between 0.1% and 20% by weight and preferably between 0.5% and 15% by weight relative to the total weight of the composition.

11. Composition according to claim 1, characterized in that the electrophilic monomer(s) is (are) chosen from the compounds of formula: 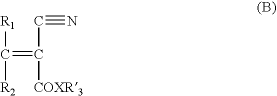
X denoting NH, S or O,
R₁ and R₂ are as defined in claim 1,
R′₃ possibly denoting a hydrogen atom or a group R as defined in claim 11.

12. Composition according to claim 11, characterized in that the monomer(s) is (are) chosen from polyfluoro(C₁₋₂₀)alkyl 2-cyanoacrylates and (C₁-C₁₀)alkyl or (C₁-C₄ alkoxy)(C₁-C₁₀ alkyl) cyanoacrylates.

13. Composition according to claim 12, characterized in that the monomer(s) is (are) chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

14. Composition according to claim 13, characterized in that the electrophilic monomer(s) correspond(s) to formula (F): 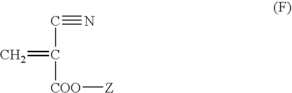
in which: Z=-(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃,
—(CH₂)₄—CH(C₂H₅)—CH₃.

15. Composition according to any one of the preceding claims, characterized in that the electrophilic monomer(s) is (are) covalently bonded to supports such as polymers, oligomers or dendrimers.

16. Composition according to any one of the preceding claims, characterized in that the amount of electrophilic monomer ranges from 0.001% to 80% by weight, preferably from 0.002% to 75% by weight and even more preferentially from 0.1% to 40% by weight relative to the total weight of the composition.

17. Composition according to any one of the preceding claims, characterized in that the cosmetically acceptable medium is anhydrous.

18. Composition according to claim 17, characterized in that the cosmetically acceptable medium is chosen from organic oils, silicones, mineral oils, plant oils, waxes, C₅-C₁₀ alkanes, acetone, methyl ethyl ketone, esters of C₁-C₂₀ acids and of C₁-C₈ alcohols, dimethoxyethane, diethoxyethane, C₁₀-C₃₀ fatty alcohols, C₁₀-C₃₀ fatty acids, C₁₀-C₃₀ fatty amides and C₁₀-C₃₀ fatty alcohol esters, and mixtures thereof.

19. Composition according to claim 18, characterized in that the cosmetically acceptable medium is chosen from olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil; polybutene oil, isononyl isononanoate, isostearyl malate, pentaerythrityl tetraisostearate, tridecyl trimellitate, and a mixture of cyclopentasiloxane/polydimethylsiloxane dihydroxylated in the α and ω positions, or mixtures thereof.

20. Composition according to any one of the preceding claims, characterized in that the composition contains polymerization inhibitors.

21. Composition according to claim 20, characterized in that the inhibitors are anionic and/or radical polymerization inhibitors.

22. Composition according to claim 20 or 21, characterized in that the polymerization inhibitors are chosen from sulfur dioxide, nitric oxide, lactone, boron trifluoride, hydroquinone and derivatives thereof such as hydroquinone monoethyl ether, tert-butylhydroquinone (TBHQ), benzoquinone and derivatives thereof such as duroquinone, catechol and derivatives thereof such as t-butylcatechol and methoxycatechol, anisole and derivatives thereof such as methoxyanisole, hydroxyanisole or butylhydroxyanisole, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans and 3-sulfonene, and mixtures thereof.

23. Composition according to any one of claims 20 to 22, characterized in that the polymerization inhibitor(s) is (are) present in an amount ranging from 10 ppm to 20% relative to the total weight of the composition.

24. Composition according to any one of the preceding claims, characterized in that the composition also contains at least one agent chosen from reducing agents, fatty substances, plasticizers, softeners, antifoams, moisturizers, pigments, clays, mineral fillers, UV-screening agents, mineral colloids, peptizers, solubilizers, fragrances, preserving agents, anionic, cationic, nonionic or amphoteric surfactants, fixing or non-fixing polymers, polyols, proteins, vitamins, direct or oxidation dyes, nacreous agents, propellants and mineral or organic thickeners.

25. Composition according to claim 24, characterized in that the agent is encapsulated.

26. Use of the composition according to any one of the preceding claims, for the cosmetic treatment of keratin fibres.

27. Use according to claim 26, for giving the hair softness, disentangling, sheen, volume and/or hold.

28. Use according to claim 26 or 27, in the presence of a nucleophilic agent.

29. Use according to claim 28, characterized in that the nucleophilic agent is chosen from molecular compounds, oligomers, dendrimers and polymers containing nucleophilic functions chosen from: R₂N⁻, NH₂⁻, Ph₃C⁻, R₃C⁻, PhNH⁻, pyridine, ArS⁻, R—C≡C⁻, RS⁻, SH, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄³¹ and H₂O, Ph representing a phenyl group; Ar representing an aryl group and R representing a C₁-C₁₀ alkyl group.

30. Use according to claim 29, characterized in that the nucleophilic agent is water.

31. Cosmetic process for treating keratin fibres, comprising the application of a composition according to any one of the preceding claims 1 to 25 to the keratin fibres in the presence of a nucleophilic agent.

32. Process according to claim 31, characterized in that the nucleophilic agent is chosen from molecular compounds, oligomers, dendrimers and polymers containing nucleophilic functions chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, Ph representing a phenyl group; Ar representing an aryl group and R representing a C₁-C₁₀ alkyl group.

33. Process according to claim 32, characterized in that the nucleophilic agent is water.

34. Process according to any one of claims 31 to 33, characterized in that the composition is applied to keratin fibres that have been moistened beforehand using an aqueous solution whose pH has been adjusted using a base, an acid or an acid/base mixture.

35. Process according to any one of claims 31 to 33, characterized in that the keratin fibres are preimpregnated with a nucleophilic agent other than water.

36. Process according to any one of claims 31 to 33, characterized in that the keratin fibres are reduced before application of the composition.

37. Process according to one of claims 31 to 36, characterized in that the application of the composition is followed by rinsing.

38. Process according to one of claims 31 to 37 characterized in that the keratin fibres are hair.

39. Cosmetic process for treating keratin fibres, comprising at least two steps, one step comprising the application of at least one amino acid other than cysteine as defined in any one of claims 2 to 9, and, with or without intermediate rinsing, another step comprising the application of at least one electrophilic monomer as defined in any one of claims 1 and 11 to 14.

40. Process according to claim 39, characterized in that the application of at least one amino acid other than cysteine is performed before the application of at least one electrophilic monomer.

41. Process according to claim 39, characterized in that the application of at least one electrophilic monomer is performed before the application of at least one amino acid other than cysteine.

42. Process according to claim 40, characterized in that it comprises the steps consisting in:
(1) applying to the hair an aqueous solution containing 0.05-40% by weight and preferably 0.1-35% by weight of amino acid(s), the percentages being expressed relative to the total weight of the solution,
(2) applying to the hair, after optional intermediate rinsing, a composition comprising 0.001-80% by weight and preferably 0.1% to 40% by weight of electrophilic monomer(s) as defined above, the percentages being expressed relative to the total weight of the composition.

43. Kit comprising a first composition containing at least one electrophilic monomer as defined in any one of claims 1 and 11 to 14, and optionally at least one anionic and/or radical polymerization inhibitor, and also a second composition comprising, in a cosmetically acceptable medium, at least one amino acid other than cysteine as defined in any one of claims 2 to 9.
